(19) United States
(12) Patent Application Publication
(10) Pub. No.: US 2020/0350077 A1
(43) Pub. Date: Nov. 5, 2020

(54) ELDERLY MORTALITY AFTER TRAUMA PREDICTION SYSTEM WITH MULTI-STAGE MODELLING AND REPORTING

(71) Applicant: Regents of the University of Minnesota, Minneapolis, MN (US)

(72) Inventors: Rachel Morris, Fox Point, WI (US); Christopher Tignanelli, Edina, MN (US); Lena M. Napolitano, Ann Arbor, MI (US); James Glover, St. Paul, MN (US); Benjamin Chen, Shoreview, MN (US)

(21) Appl. No.: 16/864,887

(22) Filed: May 1, 2020

Related U.S. Application Data
(60) Provisional application No. 62/843,123, filed on May 3, 2019.

(51) Int. Cl. G16H 50/30; G16H 10/60; G16H 50/20; G16H 50/70; G16H 15/00; A61B 5/021; A61B 5/024; A61B 5/00

(52) U.S. Cl. CPC G16H 50/30; G16H 10/60; G16H 50/20; G16H 50/70; G16H 15/00; A61B 2503/08; A61B 5/024; A61B 5/4244; A61B 5/7275; A61B 5/445; A61B 5/021

(57) ABSTRACT

An elderly-mortality prediction system includes a computing system configured to apply a first analytical model to a subset of a set of patient data parameters for computing a quick elderly mortality after trauma (qEMAT) score indicative of a likelihood of mortality of the patient, wherein the subset of the data parameters includes patient data available at admission of the patient, and further configured to apply a second analytical model to compute a full elderly mortality after trauma (fEMAT) score as an updated score indicative of a likelihood of mortality of the patient, using a full set of patient demographic data parameters including the subset of the data parameters available at admission of the patient and additional data parameters based upon diagnostic results and medical history of the patient.

This application claims the benefit of U.S. Provisional Patent Application No. 62/843,123, filed May 3, 2019, the entire content being incorporated herein by reference.

### BACKGROUND

Elderly trauma patients are often at higher risk for mortality than their younger counterparts, even when presenting with relatively minor injuries. Systems have been developed to attempt to computationally predict the likelihood of geriatric mortality in order to aid caregivers to effectively provide treatment to the patient and/or efficiently distribute medical resources. However, such systems are technically difficult and poorly utilized, often due to their reliance on data inputs which may not be available during early stages of treatment.

### SUMMARY

This disclosure describes a mortality prediction system in which a prediction engine is configured to apply analytical models that enable more rapid and accurate determination (e.g., calculated prediction) of a risk level (e.g., probability threshold) of the mortality of elderly trauma patients. Moreover, as described herein, the mortality prediction system may further recommend, based on the computed mortality risk level, a respective treatment or therapy, including, for example, resource allocation and timing.

In general, as further described herein, the mortality prediction system implements multiple, distinct analytical models to enable a two-tiered, risk-level scoring system to predict in-hospital mortality. In particular, a mortality predictor executing on a computing system of the mortality prediction system applies a first model, referred to herein as a “quick elderly mortality after trauma” (qEMAT) model, for use within the initial hours of presentation of a patient and prior to diagnostic treatment to quickly compute an initial risk level of mortality. Further, the mortality predictor subsequently applies a second model, referred to herein as a “full elderly mortality after trauma” (fEMAT) model to compute an updated risk level for use at tertiary examination using additional patient data.

The example mortality prediction systems described herein utilizing qEMAT and fEMAT models accurately estimate the probability of in-hospital mortality. For example, the qEMAT model can be used to easily calculate a qEMAT score at the time of hospital admission and/or within the first few hours, and the fEMAT model can be used later to compute an updated mortality prediction (e.g., an fEMAT score) upon obtaining additional patient data from, for example, diagnostic procedures and injury severity scoring by a treating physician. This timely information could aid in deciding transfer to tertiary referral center, patient/family counseling, and/or palliative care utilization.

In some examples, a mortality-prediction system includes a data repository configured to store one or more of a set of patient demographic data parameters for a patient having sustained one or more injuries; and a computing system configured to execute a mortality prediction engine configured to apply a first analytical model to a subset of the data parameters for computing a quick elderly mortality after trauma (qEMAT) score as an initial score indicative of a likelihood of mortality of the patient, wherein the subset of the data parameters includes only patient data available at admission of the patient, and wherein the first analytical model comprises a qEMAT analytical model configured to calculate the qEMAT score using only the subset of data parameters and without requiring, as input, a patient injury severity score (ISS) for the patient, and wherein the mortality prediction engine is further configured to apply a second analytical model to the data for computing a full elderly mortality after trauma (fEMAT) score as an updated score indicative of a likelihood of mortality of the patient, wherein the second analytical model comprises an fEMAT analytical model configured to calculate the fEMAT score using the full set of patient demographic data parameters including the subset of the data parameters available at admission of the patient and additional data parameters based upon diagnostic results and medical history of the patient.

In some examples, a method includes receiving a subset of patient demographic data parameters for a patient having sustained one or more injuries, wherein each of the subset of patient demographic data parameters is indicative of whether a particular injury, co-morbidity, or physiological condition exists for the patient, and wherein the subset of the data parameters includes only patient data parameters available at admission of the patient; applying, by a mortality prediction engine, a first analytical model to a subset of the data parameters to compute a quick elderly mortality after trauma (qEMAT) score as an initial qEMAT score indicative of a likelihood of mortality of the patient, wherein the first analytical model comprises a qEMAT analytical model configured to compute the initial qEMAT score using only the subset of data parameters and without requiring, as input, a patient injury severity score (ISS) for the patient, receiving additional patient demographic data parameters of a full set of patient demographic data parameters; applying, by the mortality prediction engine, a second analytical model to the additional patient demographic data parameters and the subset of patient demographic data parameters to compute a full elderly mortality after trauma (fEMAT) score as an updated score indicative of a likelihood of mortality of the patient, wherein the second analytical model comprises an fEMAT analytical model configured to compute the fEMAT score using the full set of patient demographic data parameters including the subset of the data parameters available at admission of the patient and additional data parameters based upon diagnostic results and medical history of the patient.

In some examples, a non-transitory computer-readable medium includes program code that, when executed, causes a processor to: store one or more of a set of patient demographic data parameters for a patient having sustained one or more injuries; apply a first analytical model to a subset of the data parameters for computing a quick elderly mortality after trauma (qEMAT) score as an initial score indicative of a likelihood of mortality of the patient, wherein the subset of the data parameters includes only patient data available at admission of the patient, and wherein the first analytical model comprises a qEMAT analytical model configured to calculate the qEMAT score using only the subset of data parameters and without requiring, as input, a patient injury severity score (ISS) for the patient, and apply a second analytical model to the data for computing a full elderly mortality after trauma (fEMAT) score as an updated score indicative of a likelihood of mortality of the patient, wherein the second analytical model comprises an fEMAT analytical model configured to calculate the fEMAT score using the full set of patient demographic data parameters including the subset of the data parameters available at admission of the patient and additional data parameters based upon diagnostic results and a medical history of the patient.

### BRIEF DESCRIPTION OF DRAWINGS

FIG. 1 is a block diagram illustrating an example computing system for predicting in-hospital mortality for patients, in accordance with one or more techniques of this disclosure.

FIG. 2 is a block diagram illustrating various example devices that may be configured to implement one or more techniques of the present disclosure.

FIGS. 3A-3D are line graphs illustrating predicted mortality rates per EMAT points, computed according to the models described herein.

FIGS. 4A-4E are example GUIs in accordance with some techniques of this disclosure.

FIG. 5 is a flowchart illustrating an example operation in accordance with the present techniques.

### DETAILED DESCRIPTION

In general, patients presenting for urgent and emergent conditions are increasingly older with more comorbidities. Unintentional injuries were recently listed as the ninth-leading cause of death by the Center for Disease and Prevention. Elderly trauma patients aged 65 or older have a higher risk for mortality after trauma. Even in the setting of equivalent injury burden, geriatric trauma patients have a higher risk of long-term mortality than their younger counterparts. Survival of elderly trauma patients is significantly affected by preexisting disease and complications, and predictive models should take these factors into account. Differences in prognostic estimates among surgeons, inadequate data about postoperative quality of life, variation in perceptions about the role of palliative care, and time constraints are contributors to surgeons providing non-beneficial operations. Elderly patients may face a long, painful, and expensive hospitalization without adding quality years to their life.

The American College of Surgeons Trauma Quality Improvement Program (ACS-TQIP) Best-Practice Guidelines state that palliative care should be provided across the entire spectrum of injury, regardless of prognosis. Special consideration for geriatric patients based on a frailty screen was recommended, but this frequently relies on the presence of a surrogate respondent. Palliative care consultation may not be able to provide services to all elderly trauma patients and their families. In order to stratify which patients would most benefit from palliative care consultation, better and more timely predicators of elderly trauma mortality are needed.

Conventional systems for predicting geriatric mortality tend to implement algorithms and models that rely on data which are often not available during early stages of treatment. For example, some mortality prediction systems utilize prognostic models that rely on the Geriatric Trauma Outcome Score (GTOS), which was developed to help make difficult decisions in complex patients using logistic regression. As recognized herein, one technical limitation of relying exclusively on these models is their reliance on an Injury Severity Score (ISS) as input, which is typically not available for weeks to months after occurrence of a patient's injury. Multiple other technical deficiencies exist. For example, the ISS is anatomically based, fails to account for physiologic status and medical comorbidities, and equally weighs high-grade head and extremity injuries. In addition, GTOS increases the score (e.g., by adding 22 points) if the patient received packed red blood cells within 24 hours, but fails to account for the number of units transfused.

In general, this disclosure describes a mortality prediction system in which a prediction engine is configured to apply analytical models that enable more rapid and accurate determination (e.g., calculated prediction) of a risk level (e.g., probability threshold) of the mortality of elderly trauma patients. Moreover, as described herein, the mortality prediction system may further recommend, based on the computed mortality risk level, a respective treatment or therapy, including, for example, resource allocation and timing.

As further described herein, the mortality prediction system implements multiple, distinct analytical models to enable a two-tiered, risk-level scoring system to predict in-hospital mortality. In particular, a mortality predictor executing on a computing system of the mortality prediction system applies a first model, referred to herein as a “quick elderly mortality after trauma” (qEMAT) model, for use within the initial hours of presentation of a patient and prior to diagnostic treatment to quickly compute an initial risk level of mortality. Further, the mortality predictor subsequently applies a second model, referred to herein as a “full elderly mortality after trauma” (fEMAT) model to compute an updated risk level for use at tertiary examination using additional patient data.

The example mortality prediction systems described herein utilizing qEMAT and fEMAT models accurately estimate the probability of in-hospital mortality. For example, the qEMAT model can be used to easily calculate a qEMAT score at the time of hospital admission and/or within the first few hours, and the fEMAT model can be used later to compute an updated mortality prediction (e.g., an fEMAT score) upon obtaining additional patient data from, for example, diagnostic procedures and injury severity scoring by a treating physician. This timely information could aid in deciding transfer to tertiary referral center, patient/family counseling, and/or palliative care utilization.

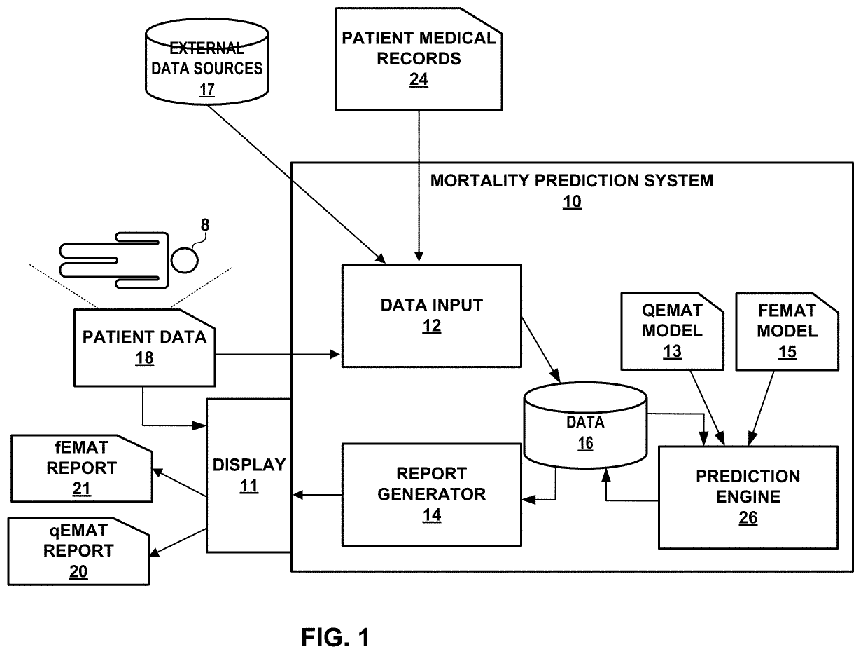

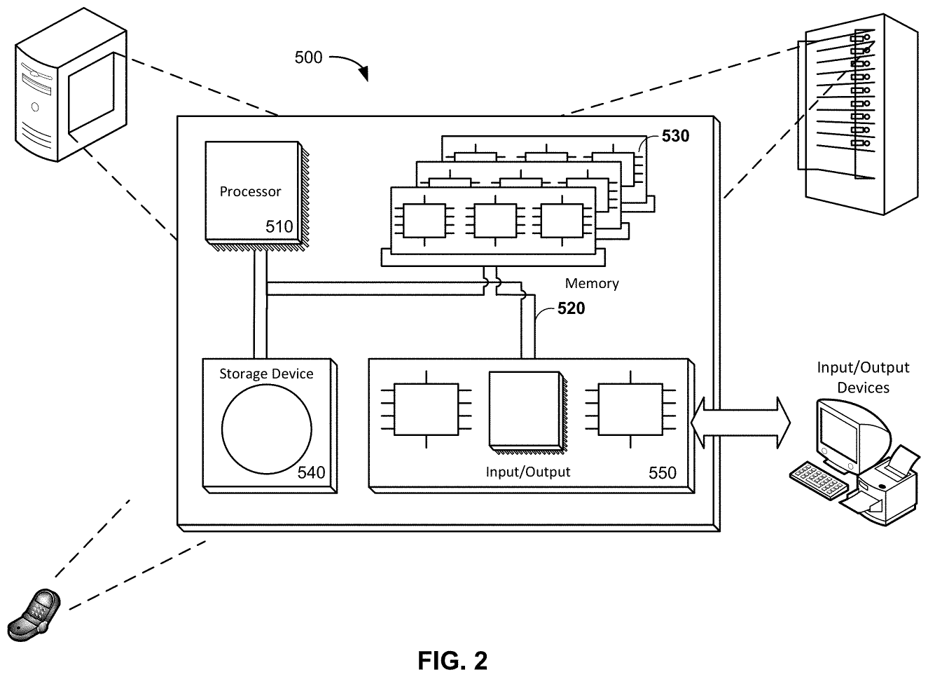

FIG. 1 is a block diagram illustrating an example computing system 10 for predicting in-hospital mortality for patients, configured according to one or more techniques of the present disclosure. As detailed below with respect to FIG. 2, system 10 may represent a computing device or computing system, such as a mobile computing device (e.g., a smartphone, a tablet computer, a personal digital assistant, and the like), a desktop computing device, a server system, a distributed computing system (e.g., a “cloud” computing system), or any other device capable of receiving patient data 18 and performing the techniques described herein.

As further described herein, mortality prediction system 10 implements multiple, distinct models to enable a two-tiered risk-level scoring system to predict in-hospital mortality. In particular, mortality predictor 26 of mortality prediction system 10 applies a “quick elderly mortality after trauma” (qEMAT) model 13 for use within hours of presentation to compute an initial risk level, referred to herein as a “qEMAT score.” Subsequently, system 10 applies a “full elderly mortality after trauma” (fEMAT) model 15 to compute an updated risk level, referred to herein as an “fEMAT score,” for use at tertiary examination.

Based on the initial risk level, mortality prediction system 10 outputs qEMAT report 20, including the qEMAT score and a corresponding recommended treatment or therapy, to aid caregivers in the early hours of care. When additional data is available, mortality prediction system 10 applies fEMAT model 15 and outputs fEMAT report 21, including the fEMAT score and a corresponding recommended treatment or therapy, to aid caregivers when providing additional care. The qEMAT score and the fEMAT score may each indicate or correspond to a quantitative probability (e.g., percent chance) or a qualitative probability (e.g., high, medium, or low) that the patient will die as a result of the traumatic injury. Each report 20, 21 may then, for example, include a suggested treatment plan selected or otherwise determined by therapy recommender 14 based on the computed mortality-risk levels (e.g., scores and/or probabilities).

In general, data input 12 receives a data indicative of a plurality of factors and criteria for patient 8. Data input 12 may, for example, query patient records 24, remote databases or systems, or other sources to automatically obtain the patient data. In addition, or alternatively, data input 12 may receive patient data manually from one or more clinicians.

Example patient data received by data input 12 and stored to data repository 16 may include data indicative of one or more of the following parameters, as non-limiting examples: patient demographics (e.g., insurance status, age, race, sex); comorbidities [e.g., myocardial infarction within the past six months, steroid use, chronic obstructive pulmonary disease (COPD), obesity, current smoker, diabetes mellitus, dementia, disseminated cancer, functionally dependent health status, cirrhosis, chronic renal failure, congestive heart failure (CHF), stroke with residual deficits]; injuries [e.g., rib fractures (1-6 rib fractures, or greater than 7 rib fractures and flail chest), hemopneumothorax, hip fracture, traumatic brain injury, pelvic fracture, femur fracture, solid organ injury (kidney, liver spleen), humerus injury, facial fracture, cervical spine fracture, thoracic/lumbar spine fracture, great vessel injury and bowel/pancreas injury]; mechanism of injury (e.g., blunt, penetrating); and most aberrant emergency-department (ED) physiologic parameters [e.g., systolic blood pressure (SBP), pulse, intubation in the ED, temperature, Glasgow Coma Scale (GCS) score (e.g., a score of 15 or lower), and respiratory rate].

System 10 may define “hypotension” using the cut-off points of systolic blood pressure less than 90 mmHg and less than 110 mmHg. The model(s) can be used with either cut-off, for example, assigning 10 points to SBP less than 110 mmHg and 13.5 points to SBP less than 90 mmHg. System 10 may define an “abnormal pulse” as a pulse greater than 120 beats per minute (BPM) or less than 50 BPM. Similarly, system 10 may define an “abnormal respiratory rate” as a respiratory rate less than 9 breaths-per-minute or greater than 29 breaths-per-minute. System 10 may dichotomize “rib fractures” into “1-6 rib fractures” and “greater than 7 rib fractures.”

Data input 12 may receive the patient data in the form of a binary “yes” or “no” input, indicating either the presence or the absence of each parameter (e.g., each independent predictor of mortality) for a particular trauma patient. Based on the received patient data, system 10 may assign a predetermined “weight” (e.g., a number of “points”) to each parameter that has been indicated to be present for the patient. An example process for determining for the value of each parameter weight is detailed further below. System 10 may then add age of the patient and the weights for each of the indicated parameters to derive the respective mortality score (e.g., qEMAT score or fEMAT score).

In various example implementations, fEMAT model 15 utilizes a full or complete set independent predictors of mortality (e.g., parameters), while qEMAT model 13 requires only a small subset of the predictors and, in particular, relies only on data available during the early stages of care, for example, at admission or within the first few hours of initial diagnosis and/or treatment. In one example, fEMAT model 15 specifies 25 independent predictors of mortality and corresponding weights as shown in the example of Table 1. These 25 parameters may include 12 injury parameters, 8 comorbidity parameters, and 5 physiologic parameters in fEMAT model 15. One or more parameters may have different weights in other examples. Weights may be adjusted based on cohorts in specific geographical regions, clinics specific experience, changes to medical procedures and/or available interventional device, etc.

TABLE 1
Patient parameters and corresponding
weight values for fEMAT score

| Patient Parameter | Weight (Points) |
|---|---|
| Injury Parameters: | |
| Bowel or pancreas injury | 20 |
| Traumatic brain injury (TBI) | 15.5 |
| Great vessel injury | 11.5 |
| Cervical spine (C-spine) injury | 11 |
| Penetrating injury | 10.5 |
| Solid organ injury | 9.5 |
| 7 or more rib fractures | 6 |
| Hemo/pneumothorax | 6 |
| Femur fracture | 5 |
| Pelvic injury | 4 |
| Thoracic/lumbar (T/L) spine injury | 1.5 |
| 1-6 rib fractures | 1 |
| Comorbidity Parameters: | |
| Advance directive with DNR in place | 23.5 |
| Cirrhosis | 17 |
| Chronic renal failure | 13 |
| Congestive heart failure (CHF) | 10 |
| Chronic obstructive pulmonary disease | 6 |
| Stroke with residual defects | 4.5 |
| Myocardial infarction(MI) (last six | 4 |
| mos.) | |
| Steroid use | 1.5 |
| Physiologic Parameters: | |
| SBP < 90 mmHg | 13.5 |
| SBP < 110 mmHg | 10 |
| Pulse > 120 bpm | 10.5 |
| Pulse < 50 bpm | 7.5 |
| (15-GCS) | 5 |
| Respiratory rate <9 or >29 | 2.5 |

The qEMAT model 13 utilizes a subset of the 25 patient parameters, for example, a subset having 8 independent predictors and corresponding weights, as shown in Table 2. As indicated, the parameter weights of the qEMAT model may in some cases be different than the parameter weights of the fEMAT model, even for similar (e.g., equivalent) parameters.

TABLE 2
Patient parameters and corresponding
weight values for qEMAT score

| Patient Parameter | Weight (Points) |
|---|---|
| SBP < 90 mmHg | 17 |
| Pulse > 120 bpm | 11.5 |
| (15-GCS) | 5.5 |
| Penetrating injury | 6 |
| Pulse < 50 bpm | 7 |
| Congestive heart failure (CHF) | 11.5 |
| Chronic renal failure | 15 |
| Cirrhosis | 19 |

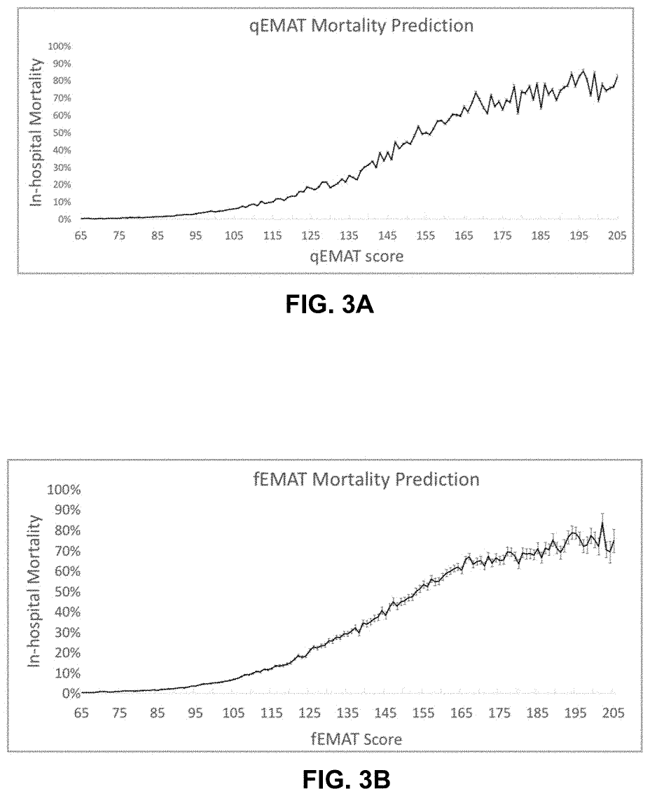

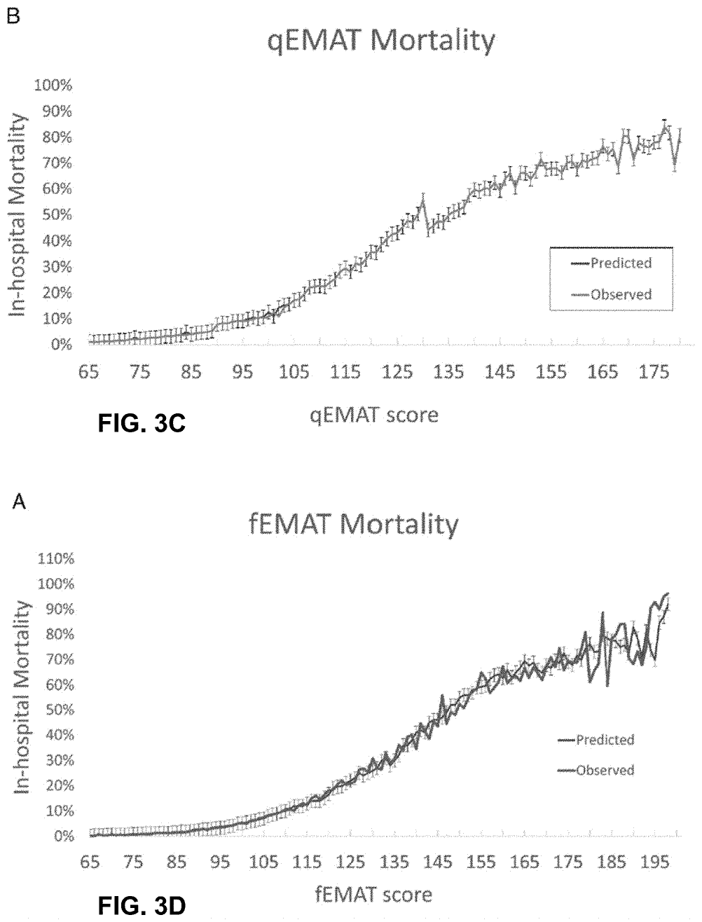

Based on the relative effect of each of these mortality-prediction parameters, e.g., using weighted averages, prediction engine 26 applies the analytical models to compute a respective score ranging from 65 points and above. For example, a minimum score of 65 points would indicate an elderly patient of age 65 having no other presenting parameters. In one example, prediction engine 26 computes the score with an integer or half-integer point scale, indicating a qEMAT or fEMAT risk level. In some examples, system 10 may translate or convert the respective mortality score into either a quantitative probability or a qualitative probability according to a predetermined correlation curve, as shown in FIGS. 3A and 3B. In some examples, system 10 may then retrieve and output a corresponding treatment or therapy recommendation based on the EMAT score or mortality probability.

FIG. 2 is a block diagram illustrating a detailed example of various devices that may be configured to implement one or more techniques of the present disclosure. That is, device 500 of FIG. 2 provides an example implementation of mortality prediction system 10 of FIG. 1 for predicting in-hospital mortality for patients. Computing device 500 may be a mobile device (e.g., a smartphone, laptop, tablet, a personal digital assistant [PDA], or other mobile device), a workstation, a computing center, a cluster of servers, or other examples of a computing environment, centrally located or distributed, that is capable of executing the techniques described herein. Any or all of the devices may, for example, implement portions of the techniques described herein for calculating a probability of mortality for elderly trauma patients. In some examples, functionality of morality prediction system 10 may be distributed across multiple computing devices, such as a cloud-based computing system for computing the predicted scores and generating the reports, and a client device, such as a tablet or mobile phone, for accessing and viewing the reports.

In the example of FIG. 2, computing device 500 includes a processor 510 that is operable to execute program instructions or software, causing the computer to perform various methods or tasks, such as performing the techniques for generating and/or using multiparametric models for prostate cancer prediction as described herein. Processor 510 is coupled via bus 520 to a memory 530, which is used to store information such as program instructions and/or other data while the computer is in operation. A storage device 540, such as a hard disk drive, nonvolatile memory, or other non-transient storage device stores information such as program instructions, data files of the multidimensional data and the reduced data set, and other information. The computer also includes various input-output elements 550, including parallel or serial ports, USB, Firewire or IEEE 1394, Ethernet, and other such ports to connect the computer to external devices such a printer, video camera, display device, medical imaging device, surveillance equipment, or the like. Other input-output elements include wireless communication interfaces such as Bluetooth, Wi-Fi, and cellular data networks.

Computing device 500 may itself be a traditional personal computer, a rack-mount or business computer or server, or any other type of computerized system. Computing device 500, in a further example, may include fewer than all elements listed above, such as a thin client or mobile device having only some of the shown elements. In another example, computing device 500 is distributed among multiple computer systems, such as a distributed server that has many computers working together to provide various functions.

FIGS. 3A-3D are graphs illustrating example predicted likelihoods of mortality using elderly-mortality-after-trauma (EMAT) models. For example, FIGS. 3A and 3C are graphs illustrating example predicted likelihoods of mortality using qEMAT model 13, and FIGS. 3B and 3D are graphs illustrating example predicted likelihoods of mortality using fEMAT model 15.

A first example of expected percentages of mortality probability for each point in a qEMAT score is depicted in FIG. 3A. In particular, FIG. 3A is a graph illustrating, based on application of qEMAT model 13, predicted likelihood of mortality for patient 8 based on the computed qEMAT risk level. As seen, the probability of in-hospital death gradually increased from 0% to 10% at 65-110 points, 20-30% at 125-136 points and 60-80% at 164-205 points (FIG. 2A). Similarly, FIG. 2B is a graph illustrating, based on a first example application of fEMAT model 15, predicted likelihood of mortality for patient 8 based on the computed fEMAT risk level. As shown in FIGS. 2A, 2B, qEMAT model 13 provides a very accurate estimation of mortality when compared with fEMAT model 15, despite using a reduced set of independent input data available during the early hours of care.

A second example of expected percentages mortality probability for each qEMAT score (in points) is depicted in FIG. 3C. In particular, FIG. 3C is a graph illustrating, based on application of qEMAT model 13 (e.g., using slightly different parameters and/or parameter weights than the example of FIG. 3A), a predicted likelihood of mortality for patient 8 (e.g., a computed qEMAT risk level). As shown in FIG. 3C, the probability of in-hospital death gradually increased from about 0% to 10% at about 65-100 points, about 10-50% at about 100-130 points, and about 50-80% at about 130-175 points.

Similarly, FIG. 3D is a graph illustrating, based on application of fEMAT model 15 (e.g., using slightly different parameters and/or parameter weights than the example of FIG. 3B), predicted likelihood of mortality for patient 8 (e.g., a computed fEMAT risk level). As shown in FIGS. 3C and 3D, qEMAT model 13 still provides a relatively accurate estimation of patient mortality when compared with fEMAT model 15, despite using a reduced set of independent input data available during the early hours of care.

In this way, mortality prediction system 10 applies distinct fEMAT model 15 and qEMAT model 13 to predict in-hospital mortality using convenient and easily available metrics to facilitate goals of care discussions and patient transfer. As an example calculation of the fEMAT score, take the hypothetical case of a 79-year-old patient with a Glasgow Coma Score (GCS) of 15, an advance directive with “Do Not Resuscitate” (DNR), chronic renal failure, 7 rib fractures, and pneumothorax who presented with a respiratory rate of 30. The fEMAT score would be equal to: [(age=79)+(advance directive=23.5)+(7 or more rib fractures=6)+(pneumothorax=6)+(chronic renal failure=13)]=127.5, which, as shown in FIG. 3D, corresponds to a predicted probability of in-hospital mortality of about 25%. Example calculations such as these may provide additional information for use as a decision aid when counseling the family and patient regarding treatment decisions.

There are multiple benefits, technical differences, and practical applications of the elderly predictive mortality models and techniques described herein. Other current systems that rely on metrics for calculating elderly mortality after trauma, including TRISS, GTOS and ISS+age, are dependent on input data that may not be available during early stages of treatment. The technical algorithms and models described herein offer significant advantages over TRISS, GTOS and ISS+age and similar techniques. Moreover, in some examples, the EMAT mortality prediction system 10 can easily be accessed and implemented through a downloadable mobile application or web-based calculator.

Further, mortality prediction system 10 implements a tiered calculator incorporating a quick score based on immediately available factors at presentation and a full score utilizing diagnostic results (e.g., imaging, measured parameter, and/or a subjective clinician evaluation) and medical history typically available much later after patient admission and initial treatment. The qEMAT score could be used as a triage tool as a comprehensive injury list is not required. qEMAT is available earlier in the hospitalization than GTOS and offers higher discrimination (AuROC) than TRISS or age+ISS. Mortality prediction system 10 may provide an accurate prediction of risk level that can be calculated much earlier in the hospitalization process without being dependent upon ISS or other data not available at that time, and therefore, can be used in immediate clinical decision-making or triage. Further, EMAT mortality prediction system 10 can be independent of ISS and AIS scoring and is able to more-specifically account for injury patterns and body regions.

The respective weight (e.g., number of points) corresponding to each patient parameter may be derived or otherwise determined by using a regression model to identify independent predictors of mortality based on a historical dataset of patient parameters and subsequent mortality rates. One example of such a regression model is a “least absolute shrinkage and selection operator” (LASSO) analysis model, with a stepwise forward-selection with p<0.01 as the cut-off for statistical significance. Selected variables may then be included in a mixed-effects model accounting for the fixed effects identified and hospital-level random effects. LASSO may be selected for parameter identification based on its calibration analysis, discrimination analysis, and ability to minimize variance. Based on the relative effect of each identified mortality predictor (e.g., a β-coefficient), the two-tiered scoring system to predict in-hospital mortality may be developed: the quick elderly mortality after trauma (qEMAT) score for use within the first hour of patient presentation and the full EMAT (fEMAT). The derived coefficients (e.g., parameter weights) may then be rounded-off to the nearest half-integer to simplify mortality score calculations. Additionally, discrimination and calibration may be assessed using the “area under Receiver Operating Characteristic” (AuROC) and Brier score, respectively.

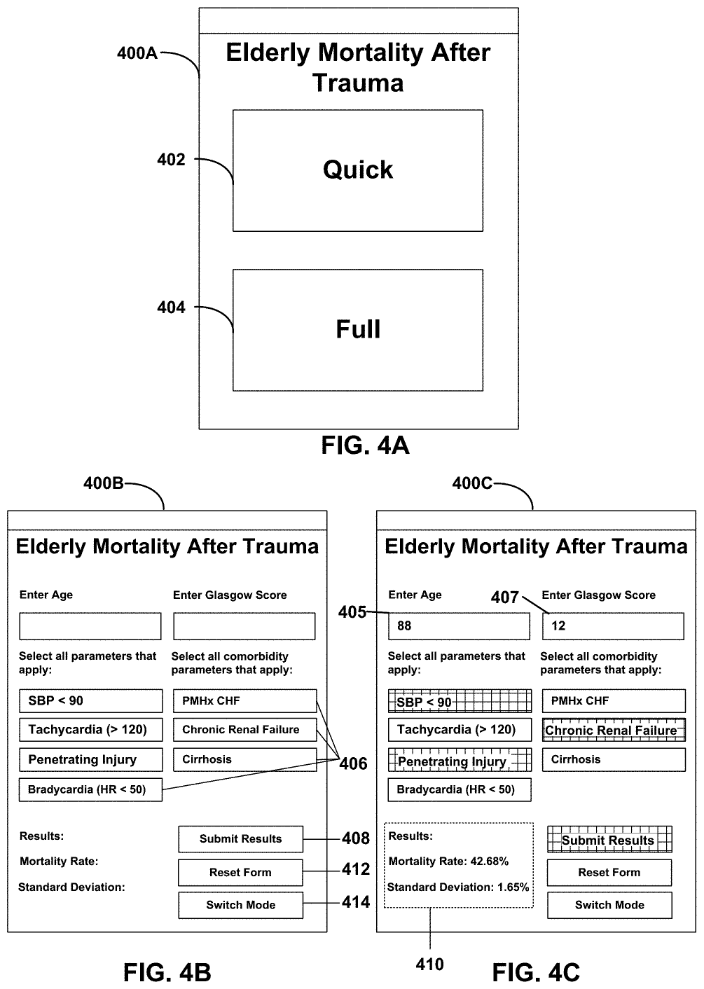

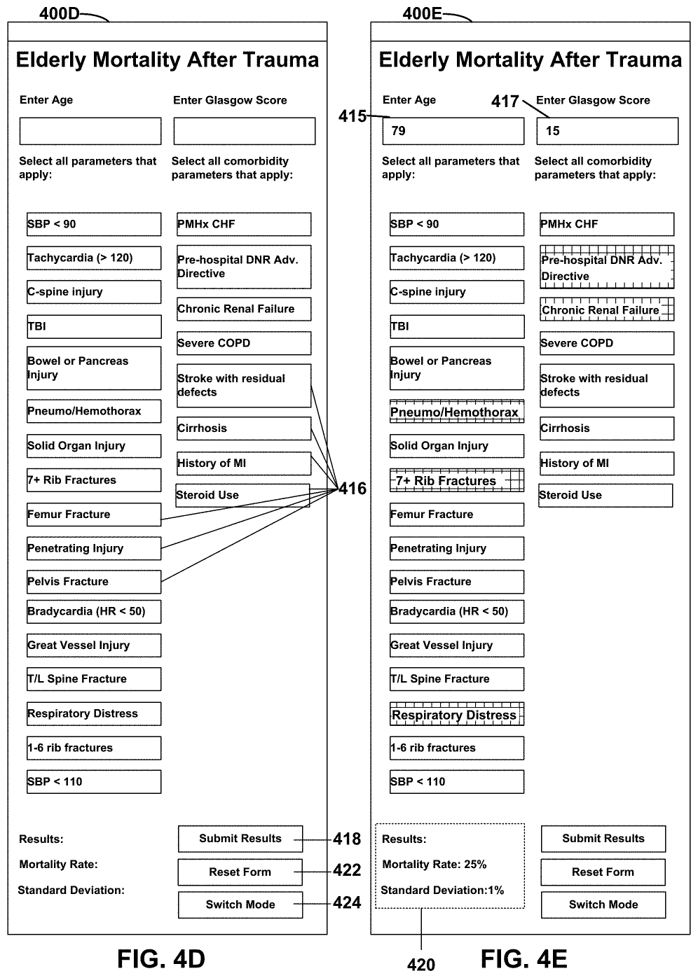

FIGS. 4A-4C are example user interfaces (UIs) 400A-400C, such as graphical user interfaces (GUIs), generated and presented by mortality prediction system 10 of FIG. 1, in accordance with some techniques of this disclosure. For example, GUIs 400A-400E may be generated and output for display on computing device 500 of FIG. 2, such as a smartphone, tablet or laptop.

FIG. 4A depicts GUI 400A. GUI 400A includes two input buttons 402, 404, allowing a user of system 10 to select between a qEMAT score calculation (“Quick” 402) and an fEMAT score calculation (“Full” 404). Selecting input button 402 may cause system 10 to display GUI 400B of FIG. 4B. Selecting input button 402 may cause system 10 to display GUI 400D of FIG. 4D.

FIG. 4B depicts example GUI 400B for capturing patient data and producing a qEMAT report 20 upon execution of qEMAT model 13 by prediction engine 26 (FIG. 1). GUI 400B includes a plurality of user input buttons 406, each indicating a different condition (e.g., injury, co-morbidity, etc.) of a patient 8, such as an elderly trauma patient. A user may select one or more buttons 406 according to information initially known about the patient. After selecting one or more inputs 406, as well as inputting the patient's age and Glasgow Coma Score, the user may select button 408 to run qEMAT model 13 to generate a qEMAT score indicating a predicted likelihood of the patient's mortality. In response, mortality prediction system 10 (FIG. 1) may generate and output for display GUI 400C of FIG. 4C.

For example, as shown in FIG. 4C, a user has input the patient's age into input box 405, the patient's Glasgow Coma Scale (GCS) score into input box 407, and selected three patient parameters 406 for a patient 8 (FIG. 1): “SBP <90,” “Chronic Renal Failure,” and a “Penetrating Injury.” The user has also actuated button 408 labeled “Submit Results.” In response, system 10 has run qEMAT model 13 to calculate, based on the selected parameters, a qEMAT report 410, indicating a predicted mortality probability of about 43%, with a standard deviation of about 1.7%. Although not depicted in FIG. 4C, in some examples, GUI 400C may also output for display a treatment or therapy recommendation based on the predicted mortality probability. The user may then select button 412 to reset the GUI (e.g., to return to GUI 400B), or may select button 414 (“Switch Mode”) to return to GUI 400A.

Once additional information is known about patient 8, e.g., via diagnostic information or the receipt of the patient's medical history, the user may select input button 404 of GUI 400A to generate GUI 400D of FIG. 4D. For example, FIG. 4D depicts example GUI 400D, for capturing patient data and producing an updated fEMAT report 21 upon execution of fEMAT model 15 by prediction engine 26 (FIG. 1). GUI 400D includes a plurality of user input buttons 416, each indicating a different condition (e.g., injury, co-morbidity, etc.) of a patient. A user may select one or more buttons 416 according to information initially known about a patient, such as an elderly trauma patient. After selecting one or more inputs, as well as inputting the patient's age and Glasgow Coma Score, the user may select button 418 to run qEMAT model and generate a qEMAT score. In response, mortality prediction system 10 (FIG. 1) may generate and output for display GUI 400E of FIG. 4E.

For example, as shown in FIG. 4E, a user has indicated a 79-year-old patient (input box 415) with a Glasgow Coma Score (GCS) of 15 (input box 417), and selected five input buttons 416 indicating (1) an advance directive with “Do Not Resuscitate” (DNR), (2) chronic renal failure, (3) 7 rib fractures, and (4) pneumothorax who presented with (5) a respiratory rate of 30 (e.g., respiratory distress). The fEMAT model would calculate an fEMAT score equal to: [(age=79)+(advance directive=23.5)+(7 or more rib fractures=6)+(pneumothorax=6)+(chronic renal failure=13)]=127.5, which, as shown in qEMAT report 420 of FIG. 4E, corresponds to a predicted probability of in-hospital mortality of about 25%. Although not depicted in FIG. 4E, in some examples, GUI 400E may also output for display a treatment or therapy recommendation based on the predicted mortality probability. The user may then select button 420 to reset the GUI (e.g., to return to GUI 400D), or may select button 422 (“Switch Mode”) to return to GUI 400A.

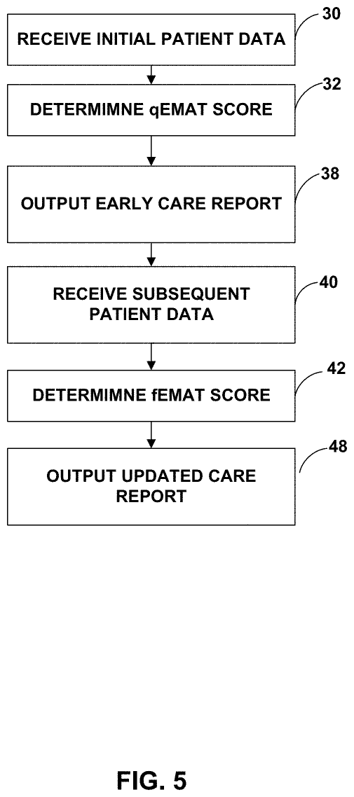

FIG. 5 is a flowchart illustrating an example operation in accordance with the techniques of this disclosure. As shown in FIG. 3, data input 12 of mortality prediction system 10 receives initial patient demographic data parameters and may store the data into data repository 16 (30). As described, initial patient data may include data indicative of one or more of the eight independent predictors of mortality utilized by qEMAT model 13 (Table 4B in the Appendix).

Based on the initial patient data, prediction engine 26 applies qEMAT model 13 to determine an initial risk level (score) indicative of a likelihood of mortality of the patient (32). For example, mortality prediction engine 26 may be configured to determine the initial qEMAT score by computing a sum of the age of the patient and the weights for any parameter of the subset of the patient demographic data parameters (e.g., particular injuries, co-morbidities, or physiological conditions) that is indicated to exist for the patient.

Utilizing the qEMAT score, report generator 14 outputs an early care report (e.g., qEMAT report 20), such as by presenting the report on display 11, which may be a local or remote display device (38). In some examples, report generator 14 may access a library of pre-defined recommended treatments and/or medical resource distribution plans and select, based on the initial predicted likelihood of mortality, one or more of the plans for inclusion within the report.

Next, data input 12 of mortality prediction system 10 receives subsequent patient data, which may be available after patient admission and initial treatment, including diagnostic data of the patient (e.g., imaging, measured parameters, and subjective physician assessments) and data entry of the patient's medical history (40). Subsequent patient data may include data indicative of one or more of the twenty-five independent predictors of mortality (e.g., patient parameters) utilized by fEMAT model 15.

Based on the additional patient data, prediction engine 26 applies fEMAT model 15 to determine an updated risk level (score) indicative of a likelihood of mortality of the patient (42). For example, mortality prediction engine 26 may be configured to determine the updated fEMAT score by computing a sum of the age of the patient and any weights for any parameter of the full set of the patient demographic data parameters (e.g., particular injuries, co-morbidities, or physiological conditions) that is indicated to exist for the patient.

Utilizing the fEMAT score, report generator 14 outputs a supplemental, updated care report (e.g., fEMAT report 21), such as by presenting the report on display 11, which may be a local or remote display device (48). In some examples, report generator 14 may select, from the library, one or more updated recommended treatment and/or medical resource distribution plans based on the updated predicted likelihood of mortality for inclusion within the report.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media, which includes any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable storage medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

What is claimed is:

1. A system comprising:
a data repository configured to store one or more of a set of patient demographic data parameters for a patient having sustained one or more injuries; and
a computing system configured to execute a mortality prediction engine configured to apply a first analytical model to a subset of the data parameters for computing a quick elderly mortality after trauma (qEMAT) score as an initial score indicative of a likelihood of mortality of the patient, wherein the subset of the data parameters includes only patient data available at admission of the patient, and wherein the first analytical model comprises a qEMAT analytical model configured to calculate the qEMAT score using only the subset of data parameters and without requiring, as input, a patient injury severity score (ISS) for the patient, and
wherein the mortality prediction engine is further configured to apply a second analytical model to the data for computing a full elderly mortality after trauma (fEMAT) score as an updated score indicative of a likelihood of mortality of the patient, wherein the second analytical model comprises an fEMAT analytical model configured to calculate the fEMAT score using the full set of patient demographic data parameters including the subset of the data parameters available at admission of the patient and additional data parameters based upon diagnostic results and medical history of the patient.

2. The system of claim 1,
wherein each of the patient demographic data parameters is indicative of whether a particular injury, co-morbidity, or physiological condition exists for the patient,
wherein the qEMAT analytical model specifies a respective first weighting for only the data parameters of the subset of patient demographic data parameters, and
wherein the fEMAT analytical model specifies a respective second weighting for each of the data parameters of the set of patient demographic data parameters.

3. The system of claim 2,
wherein the mortality prediction engine is configured to determine the initial qEMAT score by computing a sum of an age of the patient and the weights for any parameter of the subset of the patient demographic data parameters for which the particular injury, co-morbidity, or physiological condition is indicated to exist for the patient, and
wherein the mortality prediction engine is configured to determine the updated fEMAT score by computing a sum of the age of the patient and the weights for any parameter of the full set of the patient demographic data parameters for which the particular injury, co-morbidity, or physiological condition is indicated to exist for the patient.

4. The system of claim 2,
wherein the subset of patient demographic data parameters applied to the qEMAT analytical model comprises:
(1) parameters indicative of the presence or absence of a penetrating injury;
(2) parameters indicative of the presence or absence of co-morbidities including cirrhosis, chronic renal failure, and congestive heart failure; and
(3) parameters indicative of the presence or absence of the physiologic conditions of: systolic blood pressure <90 mmHg, pulse >120 bpm, pulse <50 bpm, and a Glasgow Coma Score (GCS) of 15 or lower.

5. The system of claim 2,
wherein the full set of patient demographic data parameters applied to the fEMAT analytical model comprises:
(1) parameters indicative of the presence or absence of the following injuries: bowel or pancreas injury, traumatic brain injury, great vessel injury, cervical spine injury, penetrating injury, solid organ injury, 7 or more rib fractures, hemothorax or pneumothorax injury, femur fracture, pelvic injury, thoracic/lumbar spine injury, and 1-6 rib fractures;
(2) parameters indicative of the presence or absence of the following co-morbidities: advance directive with DNR in place, cirrhosis, chronic renal failure, congestive heart failure, chronic obstructive pulmonary disease, stroke with residual defects, history of myocardial infarction within past 6 months, and steroid use; and
(3) parameters indicative of the presence or absence of the following physiologic conditions: systolic blood pressure (SBP)<90 mmHg, SBP <110; pulse >120 bpm, pulse <50 bpm, a Glasgow Coma Score (GCS) of 15 or lower, and respiratory rate <9 or >29 breaths per minute.

6. The system of claim 1, wherein the computing system comprises one or more of a cloud-based computing platform, a mobile device, a notebook computer, or a server.

7. The system of claim 1, wherein the computing system is further configured to:
determine, based on the qEMAT score, a first recommended therapy;
output, for display, the first recommended therapy;
determine, based on the fEMAT score, a second recommended therapy; and
output, for display, the second recommended therapy.

8. A method comprising:
receiving a subset of patient demographic data parameters for a patient having sustained one or more injuries, wherein each of the subset of patient demographic data parameters is indicative of whether a particular injury, co-morbidity, or physiological condition exists for the patient, and wherein the subset of the data parameters includes only patient data parameters available at admission of the patient;
applying, by a mortality prediction engine, a first analytical model to a subset of the data parameters to compute a quick elderly mortality after trauma (qEMAT) score as an initial qEMAT score indicative of a likelihood of mortality of the patient, wherein the first analytical model comprises a qEMAT analytical model configured to compute the initial qEMAT score using only the subset of data parameters and without requiring, as input, a patient injury severity score (ISS) for the patient,
receiving additional patient demographic data parameters of a full set of patient demographic data parameters;
applying, by the mortality prediction engine, a second analytical model to the additional patient demographic data parameters and the subset of patient demographic data parameters to compute a full elderly mortality after trauma (fEMAT) score as an updated score indicative of a likelihood of mortality of the patient, wherein the second analytical model comprises an fEMAT analytical model configured to compute the fEMAT score using the full set of patient demographic data parameters including the subset of the data parameters available at admission of the patient and additional data parameters based upon diagnostic results and medical history of the patient.

9. The method of claim 8,
wherein each of the patient demographic data parameters is indicative of whether a particular injury, co-morbidity, or physiological condition exists for the patient,
wherein the qEMAT analytical model specifies a respective first weighting for only the data parameters of the subset of patient demographic data parameters, and
wherein the fEMAT analytical model specifies a respective second weighting for each of the data parameters of the set of patient demographic data parameters.

10. The method of claim 9,
wherein determining the initial qEMAT score comprises computing a sum of an age of the patient and the weights for any parameter of the subset of the patient demographic data parameters for which the particular injury, co-morbidity, or physiological condition is indicated to exist for the patient, and
wherein determining the updated fEMAT score comprises computing a sum of the age of the patient and the weights for any parameter of the full set of the patient demographic data parameters for which the particular injury, co-morbidity, or physiological condition is indicated to exist for the patient.

11. The method of claim 9,
wherein the subset of patient demographic data parameters applied to the qEMAT analytical model comprises:
(1) parameters indicative of the presence or absence of a penetrating injury;
(2) parameters indicative of the presence or absence of co-morbidities including cirrhosis, chronic renal failure, and congestive heart failure; and
(3) parameters indicative of the presence or absence of the physiologic conditions of: systolic blood pressure <90 mmHg, pulse >120 bpm, pulse <50 bpm, and a Glasgow Coma Score (GCS) of 15 or lower.

12. The method of claim 9,
wherein the full set of patient demographic data parameters applied to the fEMAT analytical model comprises:
(1) parameters indicative of the presence or absence of the following injuries: bowel or pancreas injury, traumatic brain injury, great vessel injury, cervical spine injury, penetrating injury, solid organ injury, 7 or more rib fractures, hemothorax or pneumothorax injury, femur fracture, pelvic injury, thoracic/lumbar spine injury, and 1-6 rib fractures;
(2) parameters indicative of the presence or absence of the following co-morbidities: advance directive with DNR in place, cirrhosis, chronic renal failure,
congestive heart failure, chronic obstructive pulmonary disease, stroke with residual defects, history of myocardial infarction within past 6 months, and steroid use; and
(3) parameters indicative of the presence or absence of the following physiologic conditions: systolic blood pressure (SBP)<90 mmHg, SBP <110 mmHg; pulse >120 bpm, pulse <50 bpm, a Glasgow Coma Score (GCS) of 15 or lower, and respiratory rate <9 or >29 breaths per minute.

13. The method of claim 8, wherein the computing system comprises one or more of a cloud-based computing platform, a mobile device, a notebook computer, or a server.

14. The method of claim 8, further comprising:
determining, based on the qEMAT score, a first recommended therapy;
outputting for display the first recommended therapy;
determining, based on the fEMAT score, a second recommended therapy; and
outputting for display the second recommended therapy.

15. A non-transitory computer-readable medium having program code that, when executed, causes a processor to:
store one or more of a set of patient demographic data parameters for a patient having sustained one or more injuries;
apply a first analytical model to a subset of the data parameters for computing a quick elderly mortality after trauma (qEMAT) score as an initial score indicative of a likelihood of mortality of the patient, wherein the subset of the data parameters includes only patient data available at admission of the patient, and wherein the first analytical model comprises a qEMAT analytical model configured to calculate the qEMAT score using only the subset of data parameters and without requiring, as input, a patient injury severity score (ISS) for the patient, and
apply a second analytical model to the data for computing a full elderly mortality after trauma (fEMAT) score as an updated score indicative of a likelihood of mortality of the patient, wherein the second analytical model comprises an fEMAT analytical model configured to calculate the fEMAT score using the full set of patient demographic data parameters including the subset of the data parameters available at admission of the patient and additional data parameters based upon diagnostic results and a medical history of the patient.

16. The computer-readable medium of claim 15,
wherein each of the patient demographic data parameters is indicative of whether a particular injury, co-morbidity, or physiological condition exists for the patient,
wherein the qEMAT analytical model specifies a respective first weighting for only the data parameters of the subset of patient demographic data parameters, and
wherein the fEMAT analytical model specifies a respective second weighting for each of the data parameters of the set of patient demographic data parameters.

17. The computer-readable medium of claim 16,
wherein the processor is configured to determine the initial qEMAT score by computing a sum of an age of the patient and the weights for any parameter of the subset of the patient demographic data parameters for which the particular injury, co-morbidity, or physiological condition is indicated to exist for the patient, and
wherein the mortality prediction engine is configured to determine the updated fEMAT score by computing a sum of the age of the patient and the weights for any parameter of the full set of the patient demographic data parameters for which the particular injury, co-morbidity, or physiological condition is indicated to exist for the patient.

18. The computer-readable medium of claim 16,
wherein the subset of patient demographic data parameters applied to the qEMAT analytical model comprises:
(1) parameters indicative of the presence or absence of a penetrating injury;
(2) parameters indicative of the presence or absence of co-morbidities including cirrhosis, chronic renal failure, and congestive heart failure; and
(3) parameters indicative of the presence or absence of the physiologic conditions of systolic blood pressure <90 mmHg, pulse >120 bpm, pulse <50 bpm, and a Glasgow Coma Score (GCS) of 15 or lower.

19. The computer readable medium of claim 16,
wherein the full set of patient demographic data parameters applied to the fEMAT analytical model comprises:
(1) parameters indicative of the presence or absence of the following injuries: bowel or pancreas injury, traumatic brain injury, great vessel injury, cervical spine injury, penetrating injury, solid organ injury, 7 or more rib fractures, hemothorax or pneumothorax injury, femur fracture, pelvic injury, thoracic/lumbar spine injury, and 1-6 rib fractures;
(2) parameters indicative of the presence or absence of the following co-morbidities: advance directive with DNR in place, cirrhosis, chronic renal failure, congestive heart failure, chronic obstructive pulmonary disease, stroke with residual defects, history of myocardial infarction within past 6 months, and steroid use; and
(3) parameters indicative of the presence or absence of the following physiologic conditions: systolic blood pressure (SBP)<90 mmHg, SBP <110 mmHg; pulse >120 bpm, pulse <50 bpm, a Glasgow Coma Score (GCS) or lower, and respiratory rate <9 or >29 breaths per minute.

20. The computer readable medium of claim 15, wherein the processor is further configured to:
determine, based on the qEMAT score, a first recommended therapy;
output, for display, the first recommended therapy;
determine, based on the fEMAT score, a second recommended therapy; and
output, for display, the second recommended therapy.
